(19) United States
(12) Patent Application Publication
(10) Pub. No.: US 2004/0054265 A1
(43) Pub. Date: Mar. 18, 2004

(54) Psychological stress in humans

(75) Inventors: Mark Robert Cobain, Bedford (GB); Jonathan Richard Powell, Bedford (GB); Duncan Charles Stuart Talbot, Bedford (GB)

Correspondence Address: UNILEVER, PATENT DEPARTMENT, 45 RIVER ROAD, EDGEWATER, NJ, 07020, US

(73) Assignee: Conopco, Inc.

(21) Appl. No.: 10/660,125

(22) Filed: Sep. 11, 2003

(30) Foreign Application Priority Data
Sep. 13, 2002 (GB) 0221306.4

(51) Int. Cl. A61B 5/00

(52) U.S. Cl. 600/300

(57) ABSTRACT

The present invention relates to the field of psychological stress in humans, more particularly the invention relates to the diagnosis of human psychological stress through the use of isoprostanes as a biological marker.

### FIELD OF THE INVENTION

The present invention relates to the field of psychological stress in humans, more particularly the invention relates to the diagnosis of human psychological stress.

### BACKGROUND TO THE INVENTION

Psychological stress, also known as psycho-neuro-endocrine stress, involves an assessment of the biological mechanisms underlying this psychological state. The current approach is to focus on the output of the Hypothalamic-Pituitary-Adrenal (HPA) axis, since stressful events activate this system. The hypothalamus secretes corticotrophin releasing hormone (CRH) in response to stimulation. CRH then stimulates the pituitary gland to secrete adrenocorticotrophin releasing hormone (ACTH) which in turn stimulates the adrenal cortex to secrete cortisol. Increased stimulation of the HPA axis via perceived stressful events therefore results in an increased production of cortisol. The sympathetic nervous system is also activated by stress and increased activity, as measured by the output of adrenaline and noradrenaline can also be considered a marker of psychological stress.

Cortisol can be easily measured in saliva and has conventionally been used as a marker of psychological stress. Numerous cross-sectional and longitudinal studies have demonstrated the utility of measuring cortisol as a marker of stress (Seeman et al.2001, Lupien et al. 1997).

The “Allostatic Load” principle that has been developed by McEwen and Seeman (Seeman et al., 1997) suggests that repeated physiological stress puts our body at risk of long term damage. They have demonstrated this by showing that individuals with high levels of cortisol are more likely to demonstrate functional declines in physical and cognitive function.

Although an individual may be reasonably reliable at detecting their own acute stress e.g. feeling “stressed” when giving a public presentation, detection by an individual of the cumulative damage that results from repeated activation of the HPA axis over a longer period is much more difficult. It would be clearly desirable to offer a reliable means of monitoring an individuals allostatic load to increase their awareness of exposure to long term psychological stress and to thereby allow preventative steps to be taken to reduce the associated health risks.

While cortisol has been shown to be an effective marker of stress, rapid cortisol release into body fluids in response to perceived stressful events can cause large fluctuations in circulating cortisol during a daily period. Such fluctuations mask underlying and long term trends and render cortisol a poor marker of allostatic load in a single sample assessment and therefore unsuitable for identifying the individual suffering exposure to long term psychological stress.

Isoprostanes are prostaglandin like compounds which are formed in vivo by predominantly free radical catalysed non-enzymatic peroxidation of arachidonic acid. Circulating and urinary levels of F2-isoprostanes are known to be high in patients with pathologies involving oxidant stress and also in models with oxidative damage. U.S. Pat. No. 5,891,622 discloses a process for determination of oxidative stress through a comparison wherein the amount of isoprostane increase in a sample isolated from an organism undergoing oxidative stress is compared to a control. A method and kit for measuring the level of lipid peroxidation in a mammal having an oxidant stress syndrome or disease and utilising a comparative measure of an isoprostane molecular marker is disclosed in WO-A-00/32805. The prior art contains no hint or suggestion of any link between the two wholly distinct conditions of oxidative and psychological stress.

The objective technical problem addressed by the present invention is therefore to provide a process by which the damage to a human body accrued as a result of long term psychological stress can be assessed.

The solution to this problem is based on the finding that isoprostane molecules present in human biological fluids offer a reliable marker of long term psychological stress. In particular, the presence of psychological stress or increased psychological stress levels can be identified by an increase in the level of one or more isoprostanes.

### DEFINITIONS

For the purpose of the present invention an “isoprostane” refers to a free radical catalysed nonenzymic peroxidation product of arachidonic acid. These molecules are preferably selected from the group comprising F₂-isoprostanes, isoprostanes containing D/E-prostane rings (D₂-isoprostanes, E₂-isoprostanes) and isoprostanes comprising cyclopentenone ring structures (A₂-isoprostanes, J₂-isoprostanes and thrombaxane-like isoprostanes, termed isothrombaxanes (Liu et al. 1999).

The expression “F₂-isoprostane” is used herein to refer to a specific group of isoprostanes which are formed when arachidonic acid undergoes hydrogen and oxygen insertion, wherein depending on the site, four different peroxyl radical isomers are formed. Endocyclization of the radical occurs, followed by the addition of another oxygen molecule to yield endoperoxide (PGG₂-like) regioisomers. These intermediates are then reduced to F₂-IsoPs. Four regioisomers are formed and are designated as either 5-series, 12-series, 8-series or 15-series compounds depending on the carbon to which the side chain hydroxyl is attached. Each of the four regioisomers can have eight racemic diastereomers thus F₂-IsoPs for the purpose of the invention may comprise one or more of the sixty-four different compounds generated by this process (Morrow et al., 1997).

A biological marker is defined as a molecular indicator present in a sample of biological origin, wherein said indicator allows the diagnosis of a particular clinical condition to be made.

### DEFINITION OF THE INVENTION

Thus, a first aspect of the present invention provides use of one or more isoprostanes as a biological marker of psychological stress in humans.

A second aspect of the present invention provides use of a means for measuring one or more isoprostanes in determining psychological stress levels in humans.

A third aspect of the present invention provides a method of determining a psychological stress level in a human, the method comprising with measurement means, measuring the level of one or more isoprostanes in a biological sample derived from the human and calculating the psychological stress level from said measured isoprostane level.

### BRIEF DESCRIPTION OF THE INVENTION

Having hereby identified a significant positive correlation between isoprostanes and their metabolites in human body fluids and psychological stress, it is a first object of the invention to provide the use of one or more isoprostanes as a biological marker of psychological stress in humans.

A plurality of means by which isoprostane levels may be measured have been described in the art for purposes differing to that of the present invention. A second object of the invention therefore provides the use of a means for measuring one or more isoprostanes, and/or their metabolites in determining psychological stress levels in humans.

Isoprostanes may be measured in a plurality of human body fluids, in a preferred embodiment the invention comprises the use as described above wherein isoprostanes and/or their metabolites are measured in one or more fluids selected from the group comprising urine, blood, tears, sweat and saliva.

Urine has been found to offer a particularly suitable body fluid in which isoprostanes or their urinary metabolite forms of isoprostanes may be monitored. Urine that has been accumulating in the bladder for a period of more than 5 hours, more preferably for a period more than 8 hours provides a more reliable sample source. In a most preferred embodiment urine taken from overnight bladder accumulation has been found to provide most accurate results by minimising the potential influence of diet and physical activity on basal isoprostane levels.

Antibodies or antibody fragments are effective at detecting isoprostanes and therefore in a second embodiment the invention comprises a use as described above wherein said means for measuring said one or more isoprostanes comprises one or more antibodies and/or antibody fragments directed thereto.

Psychological stress is also recognised as interrelated with further psychological conditions selected from the group comprising depression, generalised anxiety disorder, post traumatic stress disorder, panic, chronic fatigue and ME. Hence, a further embodiment of the invention relates to a use according as described above, wherein said psychological stress is associated with one or more conditions selected from the group comprising clinical depression, post traumatic stress disorder, chronic fatigue syndrome and ME.

In seeking to monitor psychological stress through analysis of a urine sample it has been found that the F₂-isoprostanes are a particularly effective marker. Therefore a preferred embodiment comprises the use as described above wherein one or more F₂-isoprostanes is a 8-F₂-IsoP, as in I, and/or a racemic diastereoisomer thereof.

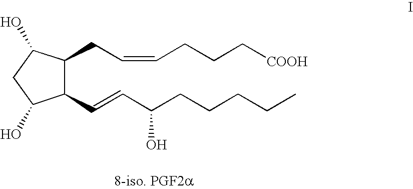

It has also been found that the isoprostane metabolite, 2,3-dinor-8-iso Prostaglandin F_{1α} is particularly effective as a biological marker of psychological stress. Therefore a further embodiment of the invention comprises the use as described above wherein said isoprostane metabolite is a 2,3-dinor-8-iso Prostaglandin F_{1α} according to the formula II (and/or a racemic diastereoisomer thereof).

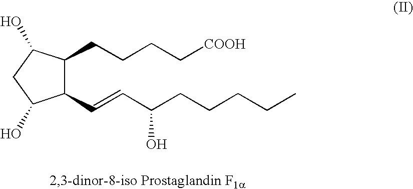

In order to help separate the effect of psychological stress on isoprostanes from other factors which may also affect or increase isoprostane levels, measurement of one or more secondary psychological stress related markers may also be determined, for example, selected from raised blood pressure, raised heart rate, increased sweating, increased tremor, levels of cortisol, adrenaline, noradrenalin and use of a questionnaire indicative of stress levels.

The present invention therefore extends to a system for determining psychological stress in humans comprising first means for effecting use or a method (respectively) according to the first, second or third aspect of the present invention and second means for determining a secondary marker of psychological stress in humans, e.g. as indicated in the preceding paragraph.

### DETAILED DESCRIPTION OF THE INVENTION

By hereby identifying biological markers of psychological stress never previously associated with this clinical condition, the invention provides clear advantage over the art. The total reliance on an individuals commonly inaccurate perception of their long term exposure to psychological stress has been removed. In its place a means of independently diagnosing this condition through a diagnostic method which measures isoprostanes levels in human tissue or body fluids is provided.

F²-isoprostanes measurement techniques for samples taken from human tissue or body fluid have been disclosed in the prior art and are commercially available. Cayman Chemicals provide suitable 8-isoprostane Enzyme Immunoassay Kits (eg Cat# 516358). US 2002/0072083 A1 of Fitzgerald et al., also discloses a means which is suitable for measuring F₂-isoprostanes and therefore can be used for the purpose of the present invention.

GC-MS remains the only other currently available method for detecting/characterising isoprostane species. However, once identified and purified (or synthesised) a similar route to the one we followed could be employed. Namely; conjugation of the selected isoprostane species to a carrier molecule, immunisation of mice and subsequent generation of specific monoclonal antibodies.

This means may comprise a sample container for carrying a tissue or body fluid sample from said mammal, a solution for use in isoprostane extraction, a negative and positive control solution for those not afflicted and afflicted respectively by psychological stress and an antibody directed against the isoprostane marker for psychological stress.

### DESCRIPTION OF FIGURES

FIG. 1: illustrates a standard curve for 8-iso PGF_{2α} detection by monoclonal 6527Ab, thereby providing the relationship between the number of counts and the inhibition by increasing the concentration of free 8-iso PGF_{2α}. The curve was used to measure the concentrations in urine samples.

FIG. 2: illustrates the correlation between urinary isoprostanes and salivary cortisol levels in humans.

FIG. 3a: illustrates the relationship between perceived psychological stress and urinary isoprostanes.

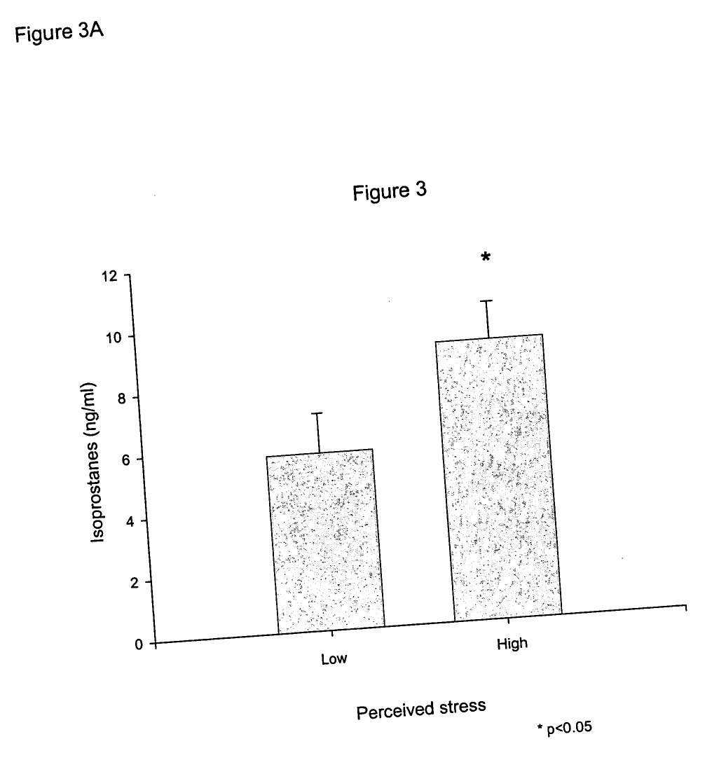

FIG. 3b: illustrates the relationship between perceived psychological stress and urinary isoprostane metabolites.

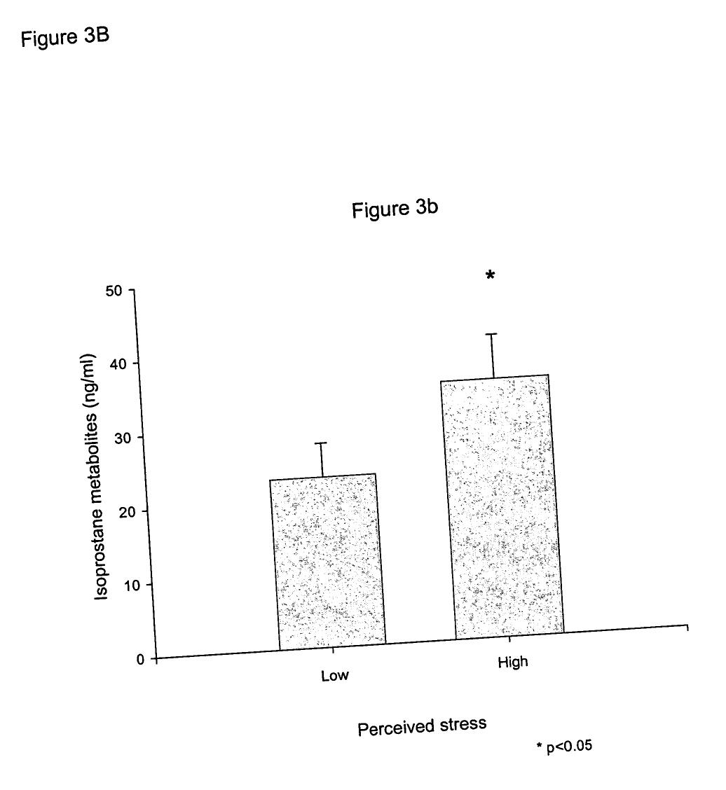

FIG. 4: illustrates the correlation between 8-F₂-IsoP and nor-adrenaline with increasing psychological stress.

References:

Liu, T., Stern, A. et al.; The Isoprostanes: Novel prostaglandin-like products of the free radical catalysed perodixation of arachidonic acid; Journal of Biomedical Science; 1999, Vol. 6, no.4 pp 226-235.

Morrow, J. D., Frei, B., Longmire, A. W. et al.; Increase in circulating products of lipid peroxidation (F-2-Isoprostanes) in smokers—smoking as a cause of oxidative damage. New England Journal of Medicine; 1995, Vol. 332, no.18, p. 1198-1203.

Morrow, J. D., Jackson Roberts, L.; The Isoprostanes: Unique bioactive products of lipid peroxidation. Pro. Lipid Res. Vol.36, No. 1, pp 1-21, 1997.

Pruessner, J. C., Helihammer, D. H., Kirschbaum, C.; Low self-esteem, induced failure and the adrenocortical stress response; Personality and Individual Differences; 1999, Vol. 27, no.3, p. 477-489.

Seeman, T. E., McEwen, B. S., ROWE, J. W. et al.; Allostatic load as a marker of cumulative biological risk: MacArthur studies of successful aging; Proceedings of the National Academy of Sciences of the United States of America; 2001, Vol. 98, no.8, p. 4770-4775.

Seeman, T. E., Singer, B. H., ROWE, J. W. et al.; Price of adaptation—Allostatic load and its health consequences—MacArthur studies of successful aging; Archives of Internal Medicine; 1997, Vol. 157, no.19, p. 2259-2268.

Cohen, S., Kamarck, T., Mermelstein, R.; A Global Measure of Perceived Stress; Journal of Health and Social Behavior; 1983, Vol. 24, p. 386-396.

Sapolsky, R. M.; Glucocorticoids, Neuroendangerment and Neurotoxicity; In: Encyclopedia of Stress; Academic Press, 2000; p. 238-244 Vol 2.

Lupien, S. J., Gaudreau, S., Tchiteya, B. M. et al.; Stress-induced declarative memory impairment healthy elderly subjects: Relationship to cortisol reactivity; Journal of Clinical Endocrinology and Metabolism; 1997, Vol. 82, no.7, p. 2070-2075.

Morrow, J. D., Roberts, L. J.; The isoprostanes: Unique bioactive products of lipid peroxidation; Progress in Lipid Research; 1997, Vol. 36, no.1, p. 1-21.

Vallieres, L., Rivest, S.; Interleukin-6 in the Central Nervous System; M-S Medecine Sciences; 2000, Vol 16, p.936-944.

### EXAMPLE 1

Preparation of Monoclonal Antibody for Isoprostane Detection:

Immunogens

8-isoPGF_{2α} purchased from Cayman Laboratories was conjugated using a number of carrier proteins (Keyhole Limpet Hemocyanin {KLH}, Bovine Serum Albumin {BSA} and Ovalbumin). The chemistry used was either carbodiimide (EDC) or mixed anhydride.

Immunisations

Female Balb/c mice were immunised subcutaneously and intra-peritoneally with 25-50 μg of appropriate immuno-conjugates. Each conjugate was mixed with an adjuvant formulation comprising aluminium hydroxide gel, saponin (Quil A) and E. coli lipopolysaccharide or Freunds. Booster injections were given on day 14 and sera from these mice were tested on day 21 to ascertain the most immunogenic conjugates. The animals with strongest immune responses were then used for fusions.

Fusions

Spleens from best-selected mice were perfused with culture media and the resulting lymphocyte suspension (approx. 10⁷ cells per spleen) was combined with SP2/ 0 myeloma cells in a 1:1 ratio. After centrifugation at 200×g for 5 minutes, the supernatant was discarded and the pellet loosened. To this mixed pellet was added 1 ml of 45% PEG 3000 (Fluka AG batch No. 251778). The cells were centrifuged gently for 3 minutes and after a total contact time of 8 minutes, the PEG was gradually diluted and removed by addition of culture medium containing selection components, Hypoxanthine and Azaserine (HAZ) and dispensed into 10×48 well plates. Hybridomas were visible at day 3 and a medium change was given on day 7. Supernatants from all fusion wells, (480 samples per fusion) were assayed for anti-8-iso PGF_{2α} activity.

Direct Assay—Enzyme Immunoassay (EIA)

Mouse antiserum and hybridoma culture supernatants were both tested by direct EIA. Greiner Microlon microtitre plates were sensitised overnight at 37° C. with 8-iso-Ovalbumin conjugate at 1 μg/ml in bicarbonate coating buffer pH 9.8. Plates were then washed with phosphate buffered saline containing 0.05% Tween 20 (PBST). 100 μl volumes of each sample were then added and the plates incubated for 1 hour at 37° C. After further washing 100 μl volumes of indicator conjugate was added (rabbit anti-mouse, alkaline phosphastase labelled {Sigma Chemicals}), followed by washing and addition of PNPP substrate. Absorbance at 405 nm was measured after approximately 20 min. incubation.

Inhibition Assay

Positive supernatants identified in direct EIA were then tested in an inhibition assay with free 8-iso PGF2α a to confirm specificity. In this assay, supernatants were first titrated to determine the dilution, which gave 70% of their maximum absorbance. These dilutions were then pre-incubated for 30 minutes with 12 doubling dilutions of inhibitor (8-iso PGF2α) from an initial concentration of 10 μg/ ml. Subsequent steps are as described in Direct assay. Related prostaglandins were then included in the inhibition assay format and the full table of cross-reactivity can be seen in Table 1.

TABLE 1

| Cross- | | | | | | | | | | | | |
|---|---|---|---|---|---|---|---|---|---|---|---|---|
| reactant | 6341 | 6363 | 6376 | 6482 | 6485 | 6478 | 6486 | 6506 | 6480.1 | 6496.1 | 6497.1 | 6527 |
| 1 | 100 | 100 | 100 | 100 | 100 | 100 | 100 | 100 | 100 | 100 | 100 | 100 |
| 2 | 3.13 | 0.3 | 5.1 | 0.07 | 0.06 | 1.09 | 0.11 | 0.61 | 1.81 | 0.38 | 0.38 | 0.1 |
| 3 | — | — | — | — | — | — | — | — | — | — | — | — |
| 4 | 2.16 | 0.26 | 1.73 | 0.23 | 1.14 | 2.39 | 1.12 | 0.45 | 2.47 | 1.66 | 1.66 | 0.21 |
| 5 | 2.78 | 0.59 | 2.91 | 0.12 | 0.35 | 1.98 | 0.53 | 3.96 | 2.67 | 1.71 | 0.45 | 3.16 |
| 6 | 0.83 | 0.11 | 0.08 | 0.83 | 5.29 | 0.63 | 7.11 | 0.15 | 0.52 | 0.3 | 7.21 | 0.13 |
| 7 | 0.13 | 0.02 | 0.05 | 0.05 | 0.01 | 0.04 | 0.01 | — | — | — | — | 0.12 |
| 8 | 1.02 | 0.11 | 0.51 | 0.37 | 1.51 | 2.23 | 2.33 | 0.09 | 3.59 | 2.01 | 2.63 | 0.62 |
| 9 | 0.85 | 0.18 | 0.07 | 0.07 | 0.02 | 0.14 | 0.01 | — | — | — | — | 0.14 |
| 10 | 0.06 | 0.03 | 0.03 | 0.03 | 0.01 | 0.01 | 0.01 | — | — | — | — | 0.09 |
| 11 | 0.08 | 0.04 | 0.06 | 0.05 | 2.46 | 1.21 | 3.09 | 0.03 | 0.49 | 0.08 | 3.47 | 0.13 |
| 12 | 0.08 | 0.06 | 0.03 | 0.26 | 2.87 | 0.24 | 3.21 | 0.04 | 0.18 | 0.14 | 4.03 | 0.13 |
| 13 | 0.21 | 0.04 | 0.08 | 0.02 | 0.02 | 0.02 | 0.01 | — | — | — | — | 0.13 |
| Approx. | 4 | 8 | 0.6 | 4 | 0.8 | 1.6 | 0.4 | 0.4 | 0.4 | 0.3 | 0.35 | 0.05 |
| sensng | | | | | | | | | | | | |
| ml | | | | | | | | | | | | |

1. 8-iso PGF2α
2. Thromboxane B2
3. Arachidonic acid
4. Prostaglandin F2α
5. 2,3-dinor-8-isoPGF1α
6. 8-isoPGE2α
7. 13,14-dihydro-15-ketoPGF2α
8. PGD2
9. 11-dihydroThromboxaneB
10. 13,14-dihydro-iS ketoPGF2a
11. 6-ketoPGFla
12. PGE2
13. 6,15-Keto 13,l4dihydroPGFlα

Table 1 shows the cross-reactivity patterns of the 12 selected antibodies. All of the antibodies give 100% cross-reactivity with 8-iso PGF_{2α} itself. The other figures are the percentage of reactivity with the related compounds. The final row is the sensitivity of each antibody to 8-iso PGF2α. ie. the concentration at which 50% inhibition is achieved. A lower figure represents a greater sensitivity.

Purified Selected Antibody Production

Clones of interest were recloned to ensure monoclonality, expanded for cryogenic preservation and used for production of purified antibody. This was achieved by growth of cells in either roller bottles or stirred fermenters up to a volume of 8 litres. Once maximum volume and optimum cell density was attained cultures were converted to serum free production. Subsequently the supernatant containing monoclonal antibody was filtered, concentrated and passed down a Protein A Sepharose column to achieve purity.

Assay Development

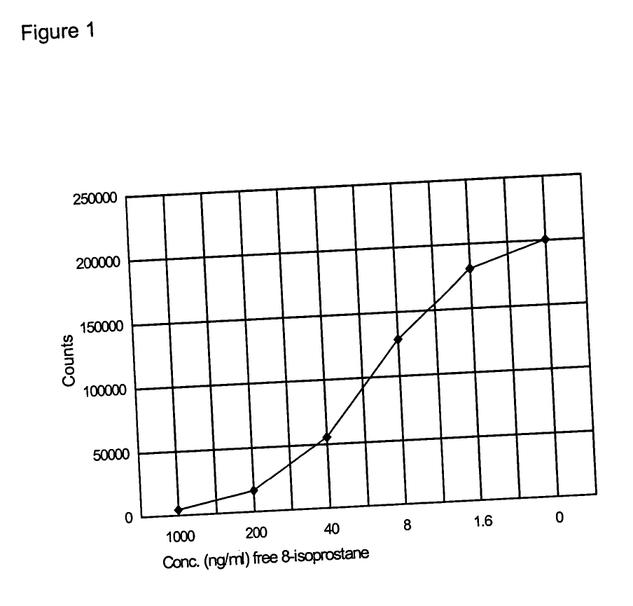

A time-resolved fluorescence immunoassay for 8-iso PGF_{2α} in urine was developed. The analysis of urine 8-iso PGF_{2α} involves competition between free 8-iso PGF_{2α} as standard or in urine and 8-iso PGF_{2α} conjugated to europium (Eu³⁺) for the immobilised antibody on the microtitre plate. The standard curve for the assay is shown in FIG. 1.

Standard DELFIA™ format 50 μl standard or sample were added to microplates coated with rabbit anti-mouse reagent (Perkin Elmer).

Antibody was diluted in DELFIA (Dissociation Enhanced Lanthanides Fluorometry Immunoassay Perkin Elmer) assay buffer and 100 μl volumes added to the microplate. 50 μl of EU³⁺ labelled 8-iso PGF_{2α} was then added at a predetermined concentration and the plate incubated shaken for 1 hour.

The assay was washed, incubated with enhancement solution and read in the Victor fluorometer. This assay determined the level of antibody and label to be used.

Subsequently, the predetermined levels of antibody and label were supplemented with known concentrations of free 8-iso PGF_{2α} as inhibitor to set up a standard curve. Clinical urine samples could then be included in the assay as unknowns and were read off the standard curve.

A similar procedure was followed to establish a monoclonal antibody to 2,3-dinor-8-isoPGF1α and a DELFIA immunoassay was developed as described above, except with the use of a 2,3-dinor labelled tracer in place of the 8-iso PGF_{2α}.

### EXAMPLE 2

Relationship Between Perceived Stress, Salivary Cortisol Response and Urinary Isoprostanes

A total of 40 participants (all female) were recruited with an average age of 33.3±5.0 years (mean±SD). 2 participants were excluded, on arrival for the study session, due to high resting heart rates (>90 beats/min).

The study consisted of two phases, firstly the filling in of a questionnaire, described below. Secondly, individuals were asked to participate in a “stress-induction” procedure designed to measure stress in a defined way in the laboratory. This allows the quantification of a biological response.(cortisol secretion) to the same stressful event in all subjects.

Stress Questionnaire

The perceived stress scale (PSS-10) is a 10 item questionnaire that has been developed by Cohen (Cohen S, Karmarck T and Mermelstein R; Global measures of perceived stress; Journal of Health and Social Behaviour; 1983, vol 24 p386-396), and is well validated. The questionnaire assesses the extent to which individuals feel that they are in control of the events in their lives. This concentration on the “locus of control” element has high face and construct validity since individuals suffering from depression report a very low sense of control over events in their lives. The sample median PSS score was calculated and subjects were characterised as high or low stress, contingent upon their position relative to that median.

Stress Induction Procedure

The procedure used was the Trier Mental Challenge Test (TMCT). This is a stressful mental arithmetic test under time pressure with negative feedback given (Kirschbaum C, Pirke KM and Helihammer DH; The TRIER social stress test—a tool for investigating psychobiological stress responses in a laboratory setting; Neurophysiology; 1993, vol 28, no.1-2, p76-81; ISSN 0302-282). It is administered using a computer program developed by Pruessner et al. (1999). It was run on a single PowerMacintosh G3 (Power PC). Participants were informed that they were performing a reaction time task to mental arithmetic sums and started with a 3 minute ‘practice’ session. This allowed the program to work out an average response time to sums. In the ‘real’ sessions (there were 3 sets of sums each lasting 4 mins) the program gave them less time than they required to complete the sums. The time limit for each sum was measured on screen by a black bar that moved across the screen and was coupled to a noise that increased in pitch as the time ran out. Participants recorded their answers by using the mouse to click on the on screen number pad. The computer would either show the word ‘correct’, ‘incorrect’ or ‘timeout’ (with the latter 2 being accompanied by a louder beep) depending on how the participant had answered the sum. At the top of the screen a bar with an arrow above and below which indicated to the participant how well they were performing in relation to a population average. They were told that they must achieve this average score in order that their reaction time data could be used.

The experimenter was present to give feedback between sets of sums to reinforce the fact that they were performing badly. It is known in the literature that the combination of internalised failure and negative feed-back during the test period induces a temporary loss of self-esteem and therefore a greater “stress response”.

Control Setting

The participants in the control setting carried out a similar task to the ‘stress’ group using the same TMCT program. In this case the ‘control setting’ was used in the program which meant there was no time limit (no black bar or noise), they had very easy sums and were not compared to other people. The experimenter gave positive feedback to the participants between sets of sums. Both the stress induction procedure and the control condition lasted 15-20 mins.

Cortisol Sampling

Prior to the test beginning, saliva samples were taken twice at ten minute intervals for analysis of cortisol levels. The average of these two samples served as a “baseline” sample for comparison between “rest” and “stress” levels. A saliva sample was taken immediately after the test and then every ten minutes post stressor for 30 minutes. This allowed for the measurement of stress for a period of time “post stress”.

Cortisol levels were measured in saliva since it is a non-invasive method and avoided any stress response as a result of having blood taken. It is a reliable way to measure free, unbound cortisol (representing the biologically active portion of cortisol) and correlates to total cortisol levels in plasma. The saliva samples were taken using “Salivettes” (supplied by Starstedt). This required participants to chew lightly on the salivette (cotton roll) for one minute or until the salivette was soaked. This is the recommended way to collect saliva for the cortisol assay used.

Measurement of salivary cortisol was performed using a high sensitivity salivary cortisol enzyme immunoassay kit from Salimetrics.

Saliva was collected using plain cotton Salivettes (Sarstedt). Samples were frozen to preciptitate mucins, thawed completely, vortexed and centrifuged at 1500 g for 15 minutes. 25 μl standard or prepared saliva sample were added in replicate to a microtitre plate coated with rabbit antibodies to cortisol. An addition of conjugate—cortisol linked to the enzyme horseradish peroxidase, is made and cortisol in the standards or samples compete with conjugate for the antibody binding sites on the plate. After an hours incubation the plate is washed to remove unbound conjugate and substrate added. The amount of bound enzyme-cortisol conjugate in the well is inversely proportional to the concentration of cortisol in the sample. The intensity of colour developed is read at 450 nm (reference 620 nm) and the measured ODs of the standards are used to construct a calibration curve against which the unknown samples are calculated.

Plates were read on a Victor 2 1420 Multilabel counter (Perkin Elmer). A Multicalc data reduction package determines individual sample concentrations from the appropriate standard curve for each assay plate.

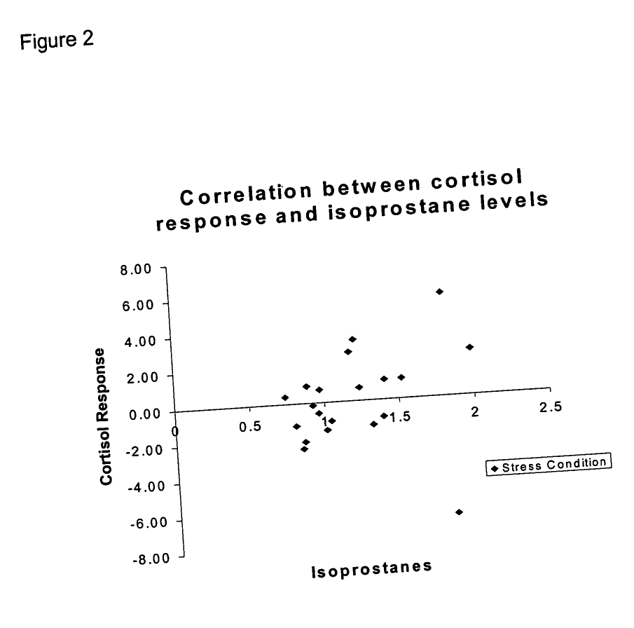

Results shown in FIG. 2.

Urinary Isoprostanes

Participants collected their first “early morning” urine sample in collection pots provided and then transferred this to a universal tube. The tube contained azide preservative and was stored, on receipt, at −20° C. An early morning sample was used in order to minimise the potential influence of diet and physical activity on basal isoprostane levels and measurements were made in accordance with example 1.

Statistical Analysis

Perceived Stress and Isoprostanes:

Perceived Stress scores were split by the sample median, therefore defining 50% of the sample as low stress and the other half as high stress. The sample median was similar to that reported in the validation paper for other populations (Cohen et al. 1985). Examination of the data indicated non-normality in the data-set. Therefore non-parametric tests were deemed appropriate and subsequently a Mann-Whitney U test was carried out to test for differences between the high and low stress groups in isoprostane levels. This revealed a significant difference between the two groups (U=110, p<0.05), with the high stress group producing increased levels of urinary isoprostanes. A significant difference was also observed in production of isoprostane metabolites between the high and low stress groups (U=106, p<0.05)The mean values of the transformed and untransformed data can be seen in Table 2.

TABLE 2
Levels of urinary isoprostanes and their metabolites for
high and low perceived stress groups. Data shown are
means (+/− s.e.m). *p < 0.05

| | Urinary isoprostanes | Urinary isoprostane |
|---|---|---|
| | (mean, ng/ml +/− s.e.m) | metabolites (mean, ng/ml +/− s.e.m) |
| High | 9.10 (+/− 1.28) | 34.93 (+/− 5.96) |
| Stress | | |
| Low | 5.78 (+/− 1.21) | 23.11 (+/− 4.56) |
| Stress | | |

Stress-Induced Cortisol and Isoprostane Levels:

In order to check that the stress induction worked, and that cortisol secretion increased as a result of the stress induction, a repeated measures analysis of variance (MANOVA) was computed with time and stress conditions as a within and between-subjects factor respectively, and with greenhouse-geiser corrections used. This demonstrated a significant time by condition interaction, such that the amount of cortisol secreted over time was greater in the stress condition than the control condition (F3,33=5.56, p=0.01).

Furthermore, in the group subjected to a stressor, there were no positive correlations between the levels of cortisol at baseline (rest) and cortisol levels after the stressor (range of correlations (r's)=31 0.13 to 0.4), indicating that baseline cortisol changed over the course of the stressor for this group. In the control group, baseline cortisol levels did positively correlate with the cortisol levels measured at each interval during the stressor (range of r's=0.6 to 0.82) indicating that these levels were stable over the time period assessed.

These results together show that cortisol secretion was altered in the stress group alone, over the course of the stress period. This reflects the increased activity of the HPA axis, indicative of increased acute stress.

Having established that the stress group was indeed demonstrating physiological signs of “stress”, we assessed the relationship between these biomarkers and the isoprostane levels. In particular the relationship with respect to the early morning urine levels. In order to investigate the relationship between isoprostanes and the extent of stress reactivity (indexed by cortisol levels during the stressor), urinary isoprostanes were correlated with the salivary cortisol levels over the timecourse of sampling. These can be seen in the table below:

TABLE 3
Correlations between urinary isoprostanes and cortisol
secreted during stress period for both “stress”
and “controls” groups. Data shown are
r-values (**p < 0.01)

| | | | Cortisol at 20 | Cortisol at 30 |
|---|---|---|---|---|
| | Cortisol at | Cortisol at 10 | mins post | mins post |
| | baseline | mins post stress | stress | stress |
| Isoprostanes | .36 | .78** | .76** | .61** |
| in Stress group | | | | |
| Isoprostanes | .21 | −.04 | −.05 | −.05 |
| in Control group | | | | |

These results demonstrate that the levels of urinary isoprostanes predict the extent to which individuals secrete more cortisol during a stressful period. There was no correlation observed between cortisol levels observed within the control group over the same period. This suggests that the influence of prior lipid peroxidation state on cortisol responsiveness is restricted to instances of physiological/psychological stress.

In total, the demonstration that reported perceived stress correlates with the isoprostane levels, and their metabolites, found in urine, and the observation that stress reactivity, as indexed by cortisol secretion occuring during a stress period, is related to the base level of isoprostanes produced indicates that isoprostanes and stress are related. It is therefore proposed that isoprostanes, as an exemplar of a biomarker of lipid peroxidation, is also a marker of the psychologically stressed state of an individual. It is further proposed that the metabolites of the isoprostanes are also related to states of psychological stress.

### EXAMPLE 3

Relationship between Urinary Noradrenaline and Urinary Isoprostanes.

In this study, 39 women in the age range 50-65 years were recruited. Participants were asked to collect their first urine sample of the morning in collection pots then transfer it to universal tube. The tubes were kept at 4° C. for less than 24 hours and then kept at −24° C. The levels of isoprostanes in the urine were measured as previously described. The measurement of noradrenaline levels in the urine were as described below.

Measurement of Noradrenaline was carried out using a CatCombi ELISA kit (www.IBL-HAMBURG.COM) distributed by RDI. These kit(s) are sandwich enzyme immunoassays which provide quantitative determination of the catecholamine, noradrenaline, in 10 μl human urine.

Urine samples firstly undergo an extraction process, followed by an incubation with more extraction buffer and an acylation reagent in macrotiter plates coated with boronate-affinity gel. Following a washing step Release buffer is placed into all wells, and the plates are stored overnight at 4° C. The extracted samples are then placed into an appropriate microtitre plate. To test for Noradrenaline, a plate coated with an antibody against N-acyl-normetanephrine is used. The plates are washed, and a second antibody directed towards a different region of the antigen molecule, conjugated to alkaline phosphatase is added. After incubation the unbound second antibody is washed off, and substrate added. The amount of bound enzyme-conjugated antibody in the well is proportional to the concentration of antigen in the sample. The intensity of colour developed is read at 490 nm (reference 620 nm) and the measured ODs of the standards are used to construct a calibration curve against which the unknown samples are calculated.

The enzyme immunoassays were read on a Victor 2 1420 Multilabel counter (Perkin Elmer). A Multicalc data reduction package determined individual sample concentrations from the appropriate standard curve for each assay plate.

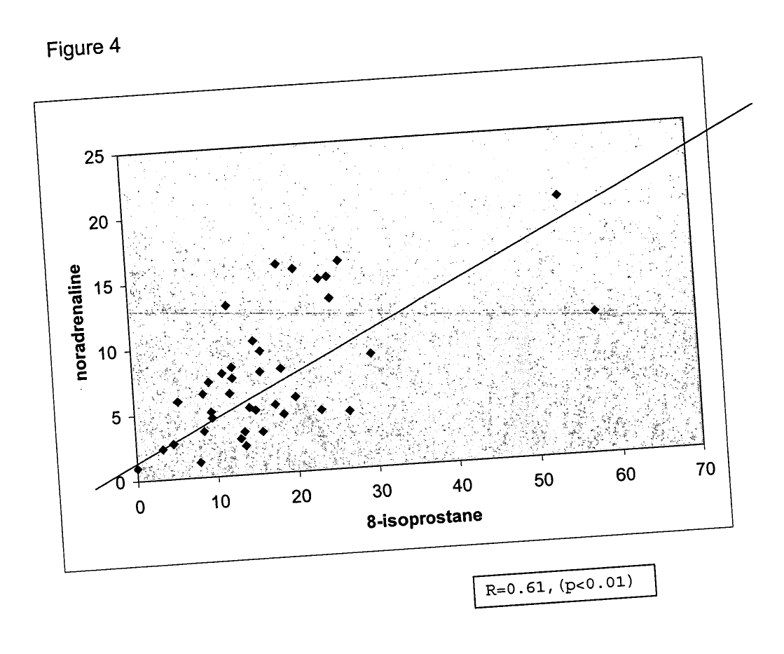

Urinary isoprostanes were measured in accordance with the previous examples. Correlational data were calculated between the levels of noradrenaline and isoprostanes measured in the overnight urine sample. These analyses revealed that noradrenaline and isoprostane levels were positively correlated, and that the correlations reached statistical significance (r=0.61,p<0.01). Scatterplots of the data are shown in FIG. 4.

1. A method of determining a pyschological stress level in a human, the method comprising with measurement means, measuring the level of one or more isoprostanes in a biological sample derived from the human and calculating the psychological stress level from said measured isoprostane level.

2. The method of claim 1, wherein said measuring means comprises means for measuring the level of one or more antibodies and/or antibody fragments directed thereto.

3. The method of claim 1, wherein said biological sample comprises one or more body fluids.

4. The method of claim 3, wherein the one or more fluids is or are selected from the group consisting of urine, blood, tears, sweat and saliva.

5. The method of claim 4, wherein said psychological stress is associated with one or more conditions selected from the group comprising clinical depression, post traumatic stress disorder, chronic fatigue syndrome and ME.

6. The method of claim 4, wherein said one or more isoprostanes when measured in urine comprises 2,3-dinor-8-iso-prostaglandin F_{1α} and/or a racemic diastereoisomer thereof.

7. The method of claim 1, wherein said one or more isoprostane is an F2-isoprostane.

8. The method of claim 1, wherein said one or more isoprostane is a 8-F₂-IsoP and/or a racemic diastereoisomer thereof.
